(19) United States
(12) Patent Application Publication
(10) Pub. No.: US 2025/0246300 A1
(43) Pub. Date: Jul. 31, 2025

(54) MEDICAL INFORMATION DISPLAY APPARATUS, IMAGE DISPLAY SYSTEM, MEDICAL INFORMATION DISPLAY METHOD, AND STORAGE MEDIUM

(71) Applicant: Konica Minolta, Inc., Tokyo (JP)

(72) Inventors: Hidekazu TAKAHASHI, Osaka (JP); Hisashi WADA, Tokyo (JP); Ken SATOU, Tokyo (JP); Kohei IKEDA, Tokyo (JP)

(21) Appl. No.: 19/025,009

(22) Filed: Jan. 16, 2025

(30) Foreign Application Priority Data
Jan. 30, 2024 (JP) 2024-011857

(51) Int. Cl. G16H 40/63; G06T 7/00; G16H 30/20

(52) U.S. Cl. CPC G16H 40/63; G06T 7/0012; G16H 30/20; G06T 2207/30096

(57) ABSTRACT

A medical information display apparatus includes a hardware processor that obtains a lesion detection process result obtained by a computer performing a lesion detection process on a medical image, and executes a prevention function of preventing user's inappropriate interpretation of the medical image.

### BACKGROUND OF THE INVENTION

### Technical Field

The present invention relates to a medical information display apparatus, an image display system, a medical information display method, and a storage medium.

### Description Of Related Art

With development of the computer technology, an apparatus having a computer-aided diagnosis (CAD) function has been used in a medical field. This function is a function of analyzing a digitally captured medical image, automatically detecting a lesion, displaying the lesion on a display, and assisting a doctor in interpretation. Further, artificial intelligence (AI) has come to be used in a lesion detection process.

At present, the lesion detection result by the AI is used as a reference for a doctor's interpretation work. Therefore, it is not necessary to wait for the AI interpretation.

However, in the future, when overtime work regulations of doctors become more stringent, the AI interpretation will become more popular, and the result of the AI interpretation will become more important. For example, even at present, in the triage type AI, the workflow is such that a doctor performs interpretation based on the primary interpretation result by the AI, and the AI is substantially responsible for the primary interpretation.

Further, when interpretation is performed in a situation in which the interpretation result by the AI does not arrive, the quality cannot be guaranteed compared to interpretation using the AI, and a doctor may overlook a lesion. Therefore, a situation in which it is necessary to wait for the interpretation result by the AI is coming.

Japanese Unexamined Patent Publication No. 2011-115265 discloses the aforementioned apparatus having a CAD and discloses displaying information indicating that a CAD's detection process result of an examination has “not arrived” on a doctor's terminal device.

### SUMMARY OF THE INVENTION

In Japanese Unexamined Patent Publication No. 2011-115265, a user who is a doctor can only know that an interpretation result has not arrived about an examination on which a lesion detection process has not finished In a case where analysis is taking time, the result is delivered if the user waits. However, in a case where the analysis fails or it is not an analysis target, the result may not be delivered even if the user waits. Therefore, a situation occurs in which it is not known whether the result will be delivered if the user waits or the result will not be delivered even if the user waits, which interferes with user's appropriate interpretation.

An object of the present invention is to assist user's appropriate interpretation.

To achieve at least one of the abovementioned objects, according to an aspect of the present invention, a medical information display apparatus reflecting one aspect of the present invention includes a hardware processor that
obtains a lesion detection process result obtained by a computer performing a lesion detection process on a medical image, and
executes a prevention function of preventing user's inappropriate interpretation of the medical image.

According to an aspect of the present invention, an image display system reflecting one aspect of the present invention includes:
an image display apparatus;
a first hardware processor that obtains a medical image, and performs a lesion detection process on the medical image; and
a second hardware processor that executes a prevention function of preventing user's inappropriate interpretation of the medical image.

According to an aspect of the present invention, a medical information display method reflecting one aspect of the present invention includes:

obtaining a lesion detection process result obtained by a computer performing a lesion detection process on a medical image; and
executing a prevention function of preventing user's inappropriate interpretation of the medical image.

According to an aspect of the present invention, a non-transitory computer-readable storage medium reflecting one aspect of the present invention stores a program causing a computer of a medical information display apparatus to:
obtain a lesion detection process result obtained by a computer performing a lesion detection process on a medical image; and
execute a prevention function of preventing user's inappropriate interpretation of the medical image.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating an overall configuration of a medical image system;

FIG. 2 is a block diagram illustrating a functional configuration of a CAD apparatus;

FIG. 3 is a block diagram illustrating a functional configuration of a server apparatus;

FIG. 4 is a block diagram illustrating a functional configuration of a viewer terminal;

FIG. 5 is a flowchart illustrating a WF information display process;

FIG. 6 is an image diagram of a WF information display screen;

FIG. 7 is an image diagram of a WF information display screen;

FIG. 8 is an image diagram of a WF information display screen; and

FIG. 9 is a flowchart illustrating a WF information display process.

### DETAILED DESCRIPTION

Hereinafter, one or more embodiments of the present invention will be described in detail with reference to the drawings. However, the scope of the invention is not limited to the embodiments or illustrated examples.

### <Configuration of Medical Image System 100>

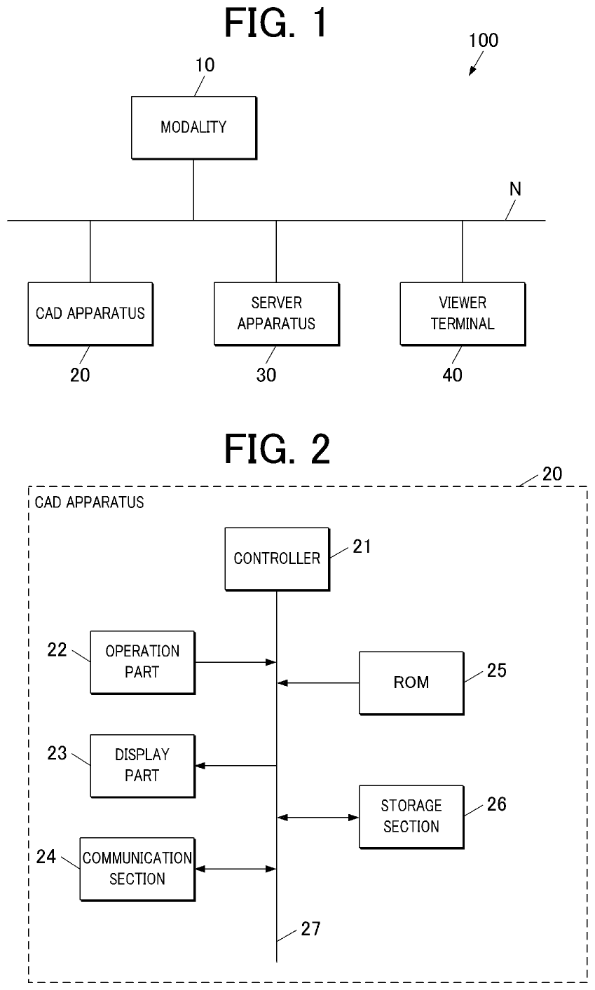

FIG. 1 illustrates a system configuration of a medical image system 100 including a viewer terminal 40 as a medical information display apparatus.

As illustrated in FIG. 1, the medical image system 100 is configured by connecting a modality 10, a CAD apparatus 20, a server apparatus 30, and a viewer terminal 40 via a network N such as a LAN (Local Area Network) so as to be able to perform data communication.

A DICOM (Digital Imaging and Communications in Medicine) standard is applied to the network N.

The modality 10 images a patient, generates a file of image data of a medical image (hereinafter referred to as an image file), and transmits the image file to each of the CAD apparatus 20 and the server apparatus 30. The image file is accompanied by supplementary information including patient information such as a patient ID, and a patient name, date of birth, and sex, and examination information such as an examination ID, an examination date, and an examination time.

As the modality 10, a computed radiography (CR) apparatus, a computed tomography (CT) apparatus, a magnetic resonance imaging (MRI) apparatus, or the like is used.

The CAD apparatus 20 receives the image file of the medical image from the modality 10, performs image analysis of the medical image, and detects a lesion. Then, the CAD apparatus 20 generates a lesion detection process result (CAD result and error information) indicating a detection result of the detected lesion.

The image analysis of the medical image may be performed using AI or the like.

The server apparatus 30 is an apparatus that manages the above-described image files and lesion detection process results.

Further, the server apparatus 30 is an apparatus that manages interpretation results of image files and lesion detection process results interpreted by a user such as a doctor using the viewer terminal 40 by executing a workflow (WF) information display process to be described later.

The viewer terminal 40 is an apparatus that displays the above-described image files and lesion detection process results. A user, such as a doctor, interprets the image files and the lesion detection process results described above and inputs interpretation results by using the viewer terminal 40.

### <Configuration of CAD Apparatus 20>

FIG. 2 illustrates a functional configuration of the CAD apparatus 20.

As illustrated in FIG. 2, the CAD apparatus 20 includes a controller 21 (first hardware processor), an operation part 22, a display part 23, a communication section 24, a ROM (Read Only Memory) 25, and a storage section 26, which are connected to each other via a bus 27.

The controller 21 includes a central processing unit (CPU), a random access memory (RAM), and the like, and comprehensively controls process operation of each component of the CAD apparatus 20.

To be specific, the CPU reads various process programs stored in the ROM 25 or the storage section 26 in response to operation signals input from the operation part 22 or instruction signals received by the communication section 24. The CPU loads the programs to a work area formed in the RAM, and performs various processes in cooperation with the programs.

The controller 21 generates a lesion detection process result (CAD result and error information), and transmits the generated CAD result to the server apparatus 30 via the communication section 24.

The controller 21 functions as an image obtaining section that obtains a medical image.

The controller 21 functions as a detection process section that performs a lesion detection process on a medical image using a computer.

The operation part 22 includes a keyboard including cursor keys, number input keys, and various function keys, and a pointing device such as a mouse, and outputs, to the controller 21, an operation signal input by a key operation on the keyboard or a mouse operation.

The display part 23 includes a liquid crystal display (LCD), and displays various screens based on display data input from the controller 21.

The communication section 24 is an interface that transmits and receives data to and from external apparatuses such as the modality 10 and the server apparatus 30.

The ROM 25 includes a nonvolatile semiconductor memory or the like, and stores a control program, parameters and files necessary for executing the program, and the like.

The storage section 26 includes a hard disk or the like, and stores various types of data.

Specifically, the storage section 26 stores a lesion detection program for detecting a lesion according to a lesion detection algorithm. Further, the storage section 26 stores image files and CAD results.

### <Configuration of Server Apparatus 30>

The server apparatus 30 is configured by a PACS (Picture Archiving and Communication System).

The server apparatus 30 stores the image file received from the modality 10, the CAD result received from the CAD apparatus 20, and the like, and provides the image file, the CAD result, and the like in response to a request from an external apparatus such as the viewer terminal 40.

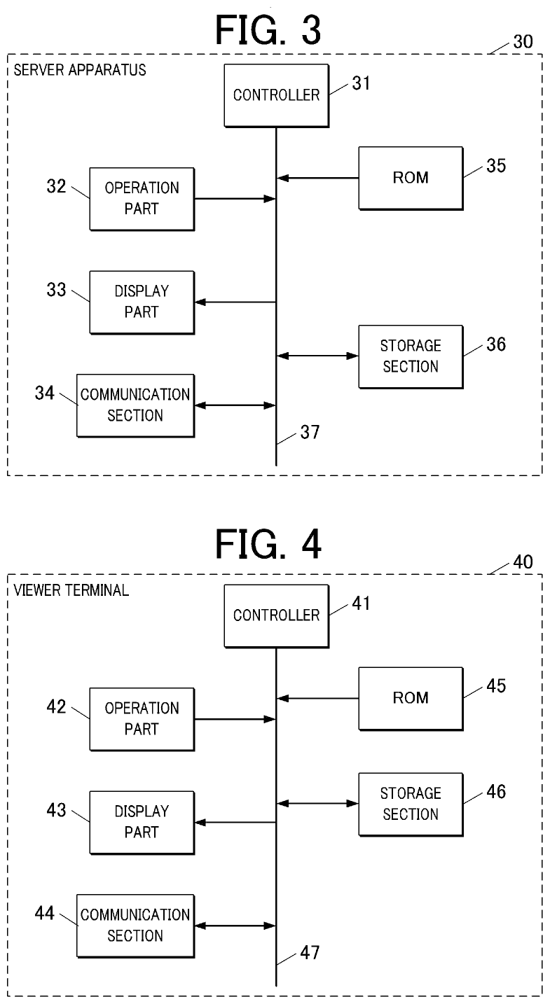

FIG. 3 illustrates a functional configuration of the server apparatus 30. As illustrated in FIG. 3, the server apparatus 30 is configured to include a controller 31, an operation part 32, a display part 33, a communication section 34, a ROM 35, and a storage section 36, which are connected to each other via a bus 37.

The controller 31 includes a CPU, a RAM and the like, and comprehensively controls process operation of each component of the server apparatus 30.

To be specific, the CPU reads various process programs stored in the ROM 35 or the storage section 36 in response to operation signals input from the operation part 32 or instruction signals received by the communication section 34. The CPU loads the programs to a work area formed in the RAM, and performs various processes in cooperation with the programs.

The controller 31 cooperates with the CAD apparatus 20 and the viewer terminal 40 to perform a WF information display process to be described later.

The operation part 32 includes a keyboard including cursor keys, number input keys, and various function keys, and a pointing device such as a mouse, and outputs, to the controller 31, an operation signal input by a key operation on the keyboard or a mouse operation.

The display part 33 includes an LCD, and displays various screens based on display data input from the controller 31.

The communication section 34 is an interface that transmits and receives data to and from external apparatuses such as the modality 10, the CAD apparatus 20, and the viewer terminal 40.

The ROM 35 includes a nonvolatile semiconductor memory or the like, and stores a control program, parameters and files necessary for executing the program, and the like.

The storage section 36 includes a hard disk or the like, and stores various types of data.

Specifically, the storage section 36 stores the image file received from the modality 10 and the CAD result received from the CAD apparatus 20 in association with each other. In addition, (a piece of) workflow (WF) information and an interpretation result are stored in the storage section 36 in association with each other.

### <Configuration of Viewer Terminal 40>

The viewer terminal 40 is a medical information display apparatus that displays a medical image based on an image file obtained from the server apparatus 30, and is an apparatus for interpretation of the medical image. The viewer terminal 40 is configured by a general PC (Personal Computer).

FIG. 4 illustrates a functional configuration of the viewer terminal 40. As illustrated in FIG. 4, the viewer terminal 40 includes a controller 41 (hardware processor, second hardware processor), an operation part 42, a display part 43 (display), a communication section 44, a ROM 45, and a storage section 46, which are connected to each other via a bus 47.

The controller 41 is composed of a CPU, a RAM and the like and comprehensively controls process operation of each component of the viewer terminal 40.

To be specific, the CPU reads various process programs stored in the ROM 45 or the storage section 46 in response to operation signals input from the operation part 42 or instruction signals received by the communication section 44. The CPU loads the programs to a work area formed in the RAM, and performs various processes in cooperation with the programs.

The controller 41 functions as an obtaining section that obtains a lesion detection process result obtained by a computer performing the lesion detection process on the medical image.

The controller 41 functions as a controller that executes a prevention function of preventing user's inappropriate interpretation of a medical image.

The operation part 42 includes a keyboard including cursor keys, number input keys, and various function keys, and a pointing device such as a mouse, and outputs, to the controller 41, an operation signal input by a key operation on the keyboard or a mouse operation.

The display part 43 includes an LCD and displays various screens based on display data input from the controller 41.

The communication section 44 is an interface that transmits and receives data to and from external apparatuses such as the server apparatus 30.

The ROM 45 includes a nonvolatile semiconductor memory or the like, and stores a control program, parameters and files necessary for executing the program, and the like.

The storage section 46 includes a hard disk or the like, and stores various types of data. For example, the storage section 46 stores an image viewer program 461 for causing the display part 43 to display a medical image when interpretation is performed.

### <WF Information Display Process>

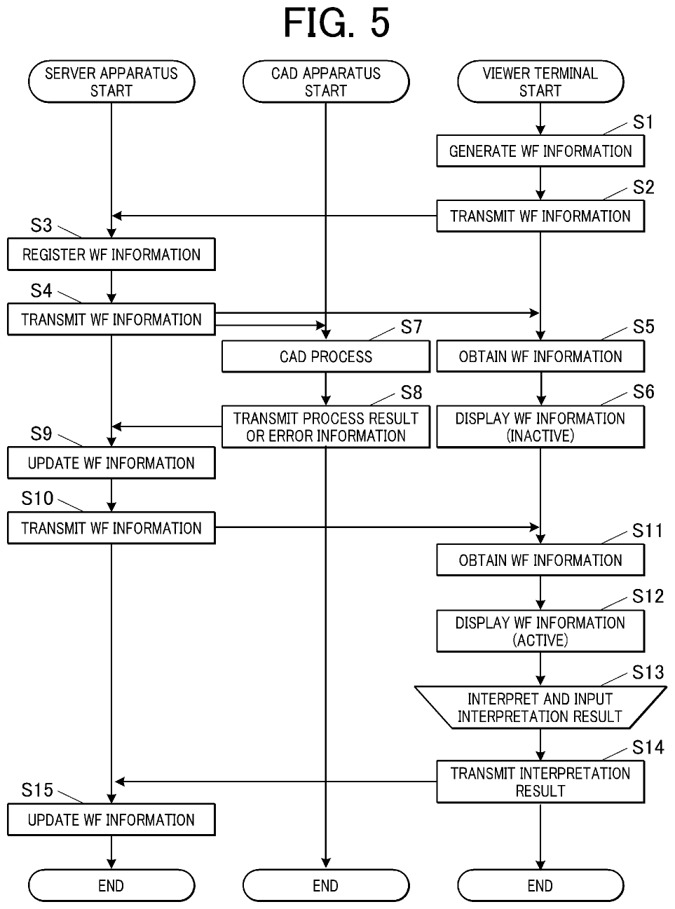

The WF information display process that is performed by the server apparatus 30, the CAD apparatus 20, and the viewer terminal 40 in cooperation with each other will be described with reference to FIG. 5.

The WF information is information indicating the order of interpreters or apparatuses that performs interpretation of a predetermined image file. Specifically, the WF information indicates the order of interpretation as follows: interpretation (lesion detection) by the CAD apparatus 20 as primary interpretation, interpretation by a user such as a doctor using the viewer terminal 40 as secondary interpretation, interpretation by another user using the viewer terminal 40 as tertiary interpretation, and so forth.

First, the controller 41 of the viewer terminal 40 generates WF information on the basis of an operation on the operation part 42 by its user (Step S1).

In the present example, the WF information is set such that the primary interpretation is interpretation by 35 the CAD apparatus 20 and the secondary interpretation is interpretation by the user such as a doctor.

Next, the controller 41 of the viewer terminal 40 transmits the WF information to the server apparatus 30 using the communication section 44 (Step S2).

Next, upon receiving the WF information using the communication section 34, the controller 31 of the server apparatus 30 associates the WF information with an image file of a medical image to be interpreted to register 40 the WF information in the storage section 36 (Step S3).

Next, the controller 31 of the server apparatus 30 transmits the WF information to the CAD apparatus 20 and the viewer terminals 40 of the user who performs the secondary interpretation using the communication section 34 (Step S4).

Next, the controller 41 of the viewer terminal 40 receives (obtains) the WF information using the communication section 44 (Step S5).

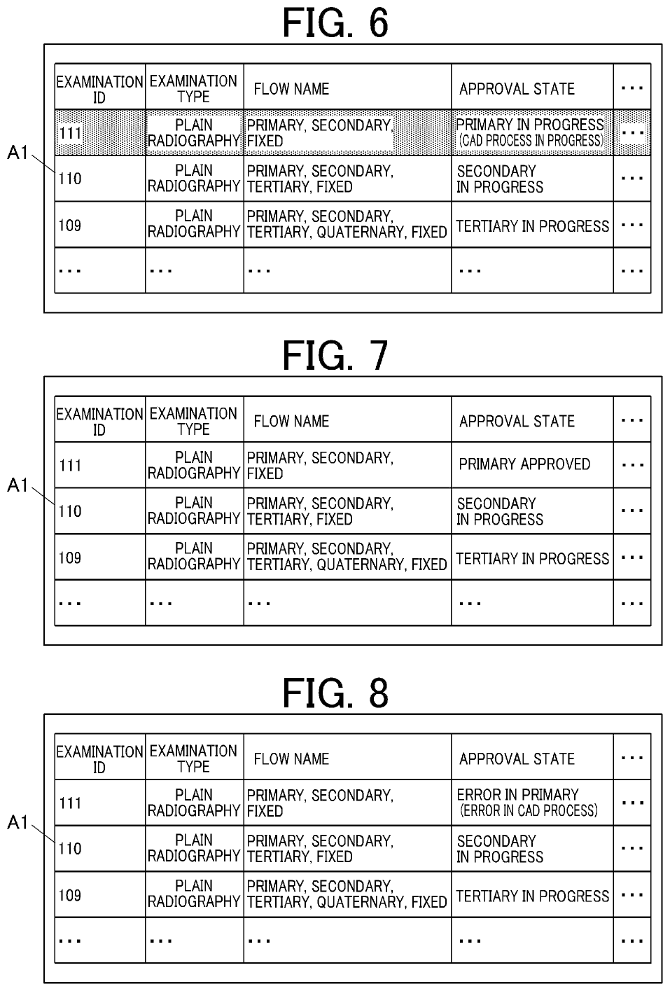

Next, the controller 41 causes the display part 43 to display a WF information display screen illustrated in FIG. 6, so that WF information is displayed (Step S6; control step).

In the present example, the interpretation by the user such as a doctor using the viewer terminal 40 is the secondary interpretation and therefore is performed after the interpretation by the CAD apparatus 20, which is the primary interpretation. Then, as illustrated in FIG. 6, the WF information is displayed in an inactive state. The inactive state will be described later. Since a CAD process (Step S7), which will be described later, is being performed in parallel, “In Analysis” or the like is displayed in an approval status field.

A WF information display screen illustrated in FIG. 6 will be described.

The area A1 is an area for displaying a list of pieces of WF information. In the area A1, when an active piece of WF information is selected and pressed (e.g., double-clicked), a screen including a medical image, patient information, supplementary information, and the like is separately displayed.

The active piece(s) of WF information can be selected and pressed, but an inactive piece(s) of WF information is set not to be selectable.

For example, in the example of FIG. 6, the piece(s) of WF information in the active state is pieces of WF information not in gray-out, and the piece(s) of WF information in the inactive state is a piece of WF information in gray-out.

By displaying the WF information display screen as described above, the user such as a doctor can check the progress status of the piece of WF information, but cannot select and press the piece of WF information, and therefore cannot start interpretation. Therefore, user's inappropriate interpretation is prevented.

Note that in the above, user's inappropriate interpretation is prevented by making the (piece of) WF information inactive, but the WF information may be made active and a warning may be displayed in a case where the WF information is selected and pressed. Even after the user sees the warning, if the user wants to start interpretation, the user may be able to start interpretation and can also advance the WF.

Next, upon receiving the WF information using the communication section 24, the CAD apparatus 20 obtains the image file associated with the WF information, and performs the CAD process (Step S7).

In a case where the CAD process is a plurality of lesion detection processes, the WF information display process proceeds to the next step after all of the lesion detection processes are completed.

Next, the CAD apparatus 20 transmits a CAD process result or error information to the server apparatus 30 using the communication section 24 (Step S8).

The error information is, for example, communication failure of a process result, system failure, or process delay due to the plurality of lesion detection processes, and is transmitted as “communication interruption/process delay/system failure”. Further, the error information is analysis failure, and is transmitted as “analysis failure/process failure”. Further, the error information indicates that the medical image is a non-target image, and is transmitted as “no analysis target/no process target”.

Next, upon receiving the CAD process result or the error information using the communication section 34, the controller 31 of the server apparatus 30 associates the CAD process result or the error information with the WF information to update the WF information (Step S9).

Next, the controller 31 of the server apparatus 30 transmits the updated WF information to the viewer terminal 40 of the user who performs the secondary interpretation using the communication section 34 (Step S10).

Next, the controller 41 of the viewer terminal 40 receives (obtains) the updated WF information using the communication section 44 (Step S11).

Next, the controller 41 causes the display part 33 to update and display the WF information as in the WF information display screen illustrated in FIG. 7 or FIG. 8 (Step S12).

Specifically, the controller 41 changes the WF information to an active state (no-gray-out state), so that the WF information can be selected and pressed. Further, in the approval state field, if a CAD process result is obtained normally in Step S7, “Primary Approved” is displayed, whereas if error information is obtained in Step S7, the error information is displayed. Display of the error information changes according to the contents of the error.

Next, when the WF information is selected and pressed (e.g., double-clicked) by the user, a screen including a medical image, patient information, supplementary information, and the like is separately displayed. The user confirms the displayed medical image, patient information, supplementary information, and the like, and performs interpretation. The user inputs an interpretation result using the operation part 42 (Step S13).

Next, the controller 41 of the viewer terminal 40 transmits the interpretation result to the server apparatus 30 using the communication section 44 (Step S14).

Next, upon receiving the interpretation result using the communication section 34, the controller 31 of the server apparatus 30 associates the interpretation result with the WF information to update the WF information (Step S15).

In the present example, the workflow ends with the secondary interpretation, and therefore, the WF information display process ends.

### <Others>

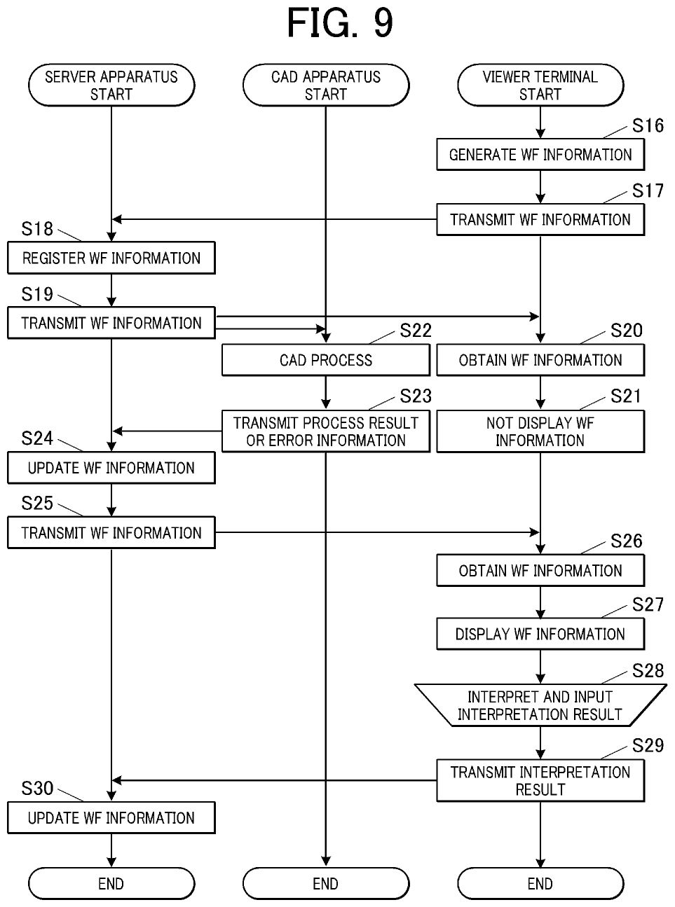

As in the WF information display process illustrated in FIG. 9, the controller 41 of the viewer terminal 40 receives (obtains) the WF information using the communication section 44 in Step S20, but may perform control to cause the display part 43 not to display the WF information on the WF information display screen in Step S21.

Then, upon receiving the updated WF information using the communication section 44 in Step S26, the controller 41 of the viewer terminal 40 may cause the display part 43 to display the WF information in Step S27, like the WF information display screen illustrated in FIG. 7 or FIG. 8.

That is, the WF information may be displayed at the timing at which interpretation is performed.

Note that each process content of the WF information display process illustrated in FIG. 9 is the same as each process content of the WF information display process illustrated in FIG. 5 having the same name, except for Step S21 and Step S27 described above.

By displaying the WF information display screen in this way, since the WF information is not displayed in the area A1, the user such as a doctor cannot select or press the WF information in the first place, and thus cannot start interpretation. Therefore, user's inappropriate interpretation is prevented.

Further, the WF information may be transmitted to the viewer terminal 40 at the timing at which interpretation is performed, without Step S5 and Step S6 of the WF information display process illustrated in FIG. 5 or Step S20 and Step S21 of the WF information display process illustrated in FIG. 9 performed. That is, in the example of FIG. 5 or the example of FIG. 9, the WF information may be received (obtained) by the viewer terminal 40 in Step S11 or Step S26.

By displaying the WF information display screen in this way, since the WF information is not displayed in the area A1, the user such as a doctor cannot select or press the WF information in the first place, and thus cannot start interpretation. Therefore, user's inappropriate interpretation is prevented.

Further, the WF information display screen may be provided with a reference button.

The reference button is a button for searching for a piece of WF information. When the reference button is pressed, a separate screen is displayed, and when an examination ID is input, search for (a piece of) WF information corresponding to the examination ID is performed.

Since the user such as a doctor can check the WF information and check the degree of progress of the CAD process, interpretation before the CAD process is completed is prevented. Further, a situation is prevented in which the user such as a doctor waits although an error has occurred in the CAD process and a CAD process result cannot be obtained.

Further, in the above, the controller 21 of the CAD apparatus 20 has the function as the detection process section that performs the lesion detection process on a medical image using a computer. However, the function as the detection process section may be provided in the server apparatus 30 or any of various information processing apparatuses connected to the network N.

Further, in the above, the primary interpretation is interpretation by the CAD apparatus 20, but not limited thereto. The effects of the present invention are exhibited as far as a doctor performs interpretation after interpretation by the CAD apparatus 20.

### EFFECTS

As described above, the medical information display apparatus (viewer terminal 40) includes the obtaining section (controller 41) that obtains a lesion detection process result (CAD result or error information) obtained by a computer performing the lesion detection process on a medical image, and the controller 41 that executes the prevention function of preventing user's inappropriate interpretation of the medical image.

Therefore, user's appropriate interpretation can be assisted.

Further, if the obtaining section (controller 41) does not obtain the lesion detection process result, the controller 41, as the prevention function, causes the display part to display information on whether to wait for the lesion detection process result to be obtained.

Therefore, user's appropriate interpretation can be assisted.

Further, as the prevention function, the controller 41 performs control to disable user's interpretation of the medical image until the lesion detection process is completed, and performs control to enable the user's interpretation after the lesion detection process is completed.

Therefore, user's appropriate interpretation can be assisted.

Further, the control to disable the user's interpretation is control that is performed by the controller 41 to cause the display part not to display an examination related to the medical image.

Therefore, user's appropriate interpretation can be assisted.

Further, the control to disable the user's interpretation is control that is performed by the controller 41 to cause the display part to display an examination related to the medical image but disable the user's interpretation to be started.

Therefore, user's appropriate interpretation can be assisted.

Further, the control to disable the user's interpretation is control that is performed by the controller 41 to cause the display part to display a warning related to the user's interpretation.

Therefore, user's appropriate interpretation can be assisted.

Further, the lesion detection process may include a plurality of lesion detection processes, and the controller 41 performs the control to disable the user's interpretation until the plurality of lesion detection processes is completed, and performs the control to enable the user's interpretation after the plurality of lesion detection processes is completed.

Therefore, user's appropriate interpretation can be assisted.

Further, the image display system (medical image system 100) is an image display system including an image display apparatus, and includes the image obtaining section (controller 21) that obtains a medical image, the detection process section (controller 21) that performs the lesion detection process on the medical image using a computer, and the controller 41 that executes the prevention function of preventing user's inappropriate interpretation of the medical image.

Therefore, user's appropriate interpretation can be assisted.

Further, the medical information display method includes an obtainment step (Step S5, Step S6, Step S20, Step S26) of obtaining a lesion detection process result obtained by a computer performing the lesion detection process on a medical image, and a control step (Step S6, Step S21) of executing the prevention function of preventing user's inappropriate interpretation of the medical image.

Therefore, user's appropriate interpretation can be assisted.

Further, the program is a program that causes a computer of the medical information display apparatus to function as the obtaining section (controller 41) that obtains a lesion detection process result obtained by a computer performing the lesion detection process on a medical image, and the controller 41 that executes the prevention function of preventing user's inappropriate interpretation of the medical image.

Therefore, user's appropriate interpretation can be assisted.

Note that those described in the above embodiment are examples of the medical information display apparatus according to the present invention, and the present invention is not limited thereto. The detailed configuration and detailed operation of the components constituting the apparatuses can be changed as appropriate without departing from the scope of the present invention.

In the above, as a computer-readable medium storing a program(s) for executing each process, a ROM, a hard disk, or the like is used as an example, but the present invention is not limited thereto. As the computer-readable medium, a nonvolatile memory, such as a flash memory, or a portable recording medium, such as a CD-ROM, can also be used. Further, a carrier wave may be used as a medium that provides data of the program(s) via a communication line.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2024-011857, filed on Jan. 30, 2024, including description, claims, drawings and abstract is incorporated herein by reference.

What is claimed is:

1. A medical information display apparatus comprising a hardware processor that
obtains a lesion detection process result obtained by a computer performing a lesion detection process on a medical image, and
executes a prevention function of preventing user's inappropriate interpretation of the medical image.

2. The medical information display apparatus according to claim 1, wherein in response to not obtaining the lesion detection process result, the hardware processor, as the prevention function, causes a display to display information on whether to wait for the lesion detection process result to be obtained.

3. The medical information display apparatus according to claim 1, wherein the hardware processor
as the prevention function, performs control to disable user's interpretation of the medical image until the lesion detection process is completed, and
after the lesion detection process is completed, performs control to enable the user's interpretation.

4. The medical information display apparatus according to claim 3, wherein the control to disable the user's interpretation is control that is performed by the hardware processor to cause a display not to display an examination related to the medical image.

5. The medical information display apparatus according to claim 3, wherein the control to disable the user's interpretation is control that is performed by the hardware processor to cause a display to display an examination related to the medical image but disable the user's interpretation to be started.

6. The medical information display apparatus according to claim 3, wherein the control to disable the user's interpretation is control that is performed by the hardware processor to cause a display to display a warning related to the user's interpretation.

7. The medical information display apparatus according to claim 3,
wherein the lesion detection process includes a plurality of lesion detection processes, and
wherein the hardware processor
performs the control to disable the user's interpretation until the plurality of lesion detection processes is completed, and
performs the control to enable the user's interpretation after the plurality of lesion detection processes is completed.

8. An image display system comprising:
an image display apparatus;
a first hardware processor that obtains a medical image, and performs a lesion detection process on the medical image; and
a second hardware processor that executes a prevention function of preventing user's inappropriate interpretation of the medical image.

9. A medical information display method comprising:
obtaining a lesion detection process result obtained by a computer performing a lesion detection process on a medical image; and
executing a prevention function of preventing user's inappropriate interpretation of the medical image.

10. A non-transitory computer-readable storage medium storing a program causing a computer of a medical information display apparatus to:
obtain a lesion detection process result obtained by a computer performing a lesion detection process on a medical image; and
execute a prevention function of preventing user's inappropriate interpretation of the medical image.

11. The non-transitory computer-readable storage medium according to claim 10, wherein the program causes the computer of the medical information display apparatus to, in response to not obtaining the lesion detection process result, as the prevention function, cause a display to display information on whether to wait for the lesion detection process result to be obtained.

12. The non-transitory computer-readable storage medium according to claim 10, wherein the program causes the computer of the medical information display apparatus to:
as the prevention function, perform control to disable user's interpretation of the medical image until the lesion detection process is completed, and
after the lesion detection process is completed, perform control to enable the user's interpretation.

13. The non-transitory computer-readable storage medium according to claim 12, wherein the control to disable the user's interpretation is control that is performed by the computer of the medical information display apparatus to cause a display not to display an examination related to the medical image.

14. The non-transitory computer-readable storage medium according to claim 12, wherein the control to disable the user's interpretation is control that is performed by the computer of the medical information display apparatus to cause a display to display an examination related to the medical image but disable the user's interpretation to be started.

15. The non-transitory computer-readable storage medium according to claim 12, wherein the control to disable the user's interpretation is control that is performed by the computer of the medical information display apparatus to cause a display to display a warning related to the user's interpretation.

16. The non-transitory computer-readable storage medium according to claim 12,
wherein the lesion detection process includes a plurality of lesion detection processes, and
wherein the program causes the computer of the medical information display apparatus to:
perform the control to disable the user's interpretation until the plurality of lesion detection processes is completed; and
perform the control to enable the user's interpretation after the plurality of lesion detection processes is completed.
